(19) United States
(12) Patent Application Publication
(10) Pub. No.: US 2008/0241870 A1
(43) Pub. Date: Oct. 2, 2008

(54) Composition For Creating an Artificial Bone Marrow Like Environment and Use Thereof

(75) Inventors: Vaijayanti Prakash Kale, Pune (IN); Lakshmi Charan Padhy, Mumbai (IN)

Correspondence Address: MERCHANT & GOULD PC, P.O. BOX 2903, MINNEAPOLIS, MN, 55402-0903, US

(73) Assignee: NATIONAL CENTRE FOR CELL SCINECES, PUNE (IN)

(21) Appl. No.: 11/817,173

(22) PCT Filed: Jul. 22, 2005
(86) PCT No.: PCT/IB05/02249

§ 371 Date: May 30, 2008

(30) Foreign Application Priority Data
Mar. 1, 2005 (IN) 230/MUM2005

(51) Int. Cl. C12Q 1/02; C12N 5/06

(52) U.S. Cl. 435/29; 435/395; 435/366; 435/375

(57) ABSTRACT

The present invention is in the domain of cell biology and medicine and relates to composition and in vitro methods for creation of artificial bone-marrow like environment and uses thereof.

### FIELD OF INVENTION

The present invention is in the domain of cell biology and medicine and relates to composition and in vitro methods for creation of artificial bone-marrow like environment and uses thereof.

### BACKGROUND ART

In humans, a specialized environment termed as “Bone marrow micro-environment” (BME) exists within the bone cavity that constitutes the principal site for the formation of at least ten different types of blood cells and cells necessary for rejuvenation of bone and a host of stem/progenitor cells that are capable of giving rise to a wide variety of differentiated cells. Human health is critically dependent on a continual supply of different blood cells that are produced by a process called “hematopoiesis”. BME may direct a small number of pluripotent stem and progenitor cells (SPC) that may circulate within the body to generate as many as ten different kinds of mature blood cells comprising the overall hematopoietic process. Since SPC are widely distributed within the body, natural mechanisms operate to a) allow SPC from outside the bone marrow to home in to the BME (called SPC-Homing), b) to retain SPC within the BME by promoting suitable adhesive interactions between the SPC and the BME (called engraftment), c) to allow transition of quiescent SPC to an activated form to foster their proliferation, d) to allow SPC to survive against apoptosis in the face of a multitude of often conflicting signals ( called survival of SPC), e) to instruct SPC to proliferate along the pathways of either self renewal (to produce more of SPC) or lineage commitment and differentiation (to produce more of mature blood cells). The role of BME on Hematopoiesis involving SPC can therefore be construed as a series of steps which are individually regulated and delicately balanced so as to ensure efficient multi-lineage blood cell formation throughout the life time of an individual. The entire process relating to SPC functioning (homing, engraftment, activation from quiescence, induction of quiescence on activated SPC, cell-survival, self renewal, lineage commitment and proliferation along differentiation) leading to sustained and optimal blood cell production is regulated by the BME but the exact mechanism involved in this regulation is not fully understood by scientists and there is no method in the prior art for the creation of a BME-like environment to regulate one or more of the steps enumerated above in vitro or in vivo and to enable a variety of other uses.

So far it was believed in the prior art that the accessory cells present in the bone marrow only contribute the various cytokines and growth regulators in their immediate neighborhoods for the formation of the micro-environment required by SPC and thus function as a passive source of such components required for hematopoiesis. Another view point prevalent in the literature is that only a rare population of accessory cells has the ability to support SPC development. The applicant has however gone against such thinking of the prior art and found that unselected accessory cells may provide superior signals to SPC if provided an appropriate condition. The applicant has now developed a composition that assists in developing an artificial bone marrow like environment where blood cell formation may be enhanced or regulated. The composition and methods disclosed by the invention is such that all the steps of hematopoiesis may be substantially improved. Furthermore, the invention underscores that a brief contact of SPC with the accessory cells or cells comprising the ABME is required for the desired effects to manifest and hence emphasizes that the accessory cells do play an active role. Thus, in the prior art, attempts have been made to expand SPC. However, so far it has not been possible to substantially improve or regulate blood cell formation by creating an effective artificial BME in vitro. The present invention fulfils this need.

### OBJECTS OF THE INVENTION

The main object of the invention is to provide a composition that assists in developing an artificial bone marrow like environment where blood cell formation may be enhanced or regulated.

Another object is to provide a method for preparing such an environment.

### DESCRIPTION OF THE ACCOMPANYING DRAWINGS

FIGS. 1A to 1D: FIG. 1 shows formation of hematopoietic colonies when mononuclear cells were plated in different types of media.

FIGS. 2A-2E: FIG. 2B represents the outcome of a colony formation assay by mixing MNC with mesenchymal cells, 2C, 2D and 2E after including the contacting step respectively with Biological, Chemical or Immunological hematomodulators with the mesenchymal cells. 2A shows the nature of colonies formed only from MNC without the mesenchymal cells.

FIGS. 3A to 3B show that equipotent ABME are formed with the use of TGF β1 and FGF-2 respectively when used separately.

FIGS. 4A to 4E shows the effect of various hematopoietic modulators on colony formation and development of hematopoietic cells.

FIG. 5 shows the effect of an Immunological hematomodulator and its efficacy.

FIGS. 6(a-c) shows that the mesenchymal cells present in ABME express increased amounts of chemotactic molecules like SDF1α when they are treated with suitable hematomodulators, thereby resulting in enhanced migration or homing and adherence FIGS. 6(d-f) of SPC.

FIG. 7 shows modulation of hematopoiesis by choosing appropriate combination of different hematomodulators. The bars represent the hematopoietic colonies formed when a fixed number of MNC are used and the ABME formed on mesenchymal cells by using both an activating and an inhibiting type of hematomodulator.

FIGS. 8(a-b) shows alteration of lineage commitment of HSPC by contacting them with ABME created with appropriate hematomodulators.

FIGS. 9(a-d) shows increase in cell survival factors in ABME by treating them with suitable hematomodulators.

FIGS. 10(a-b) shows increase in self renewal divisions in SPC by using suitable hematomodulators. FIG. 10(c) shows the increased expression of a signaling molecule Jagged 1 in the prepared ABME cells that supports self renewal.

FIGS. 11(a-b) shows creation of hypoxic environment in cells under normoxic conditions by the use of suitable hematomodulators.

FIG. 12 shows assessment of relative efficacy of two or more given mesenchymal populations to form ABME when contacted with a given hematomodulator.

FIG. 13 shows screening for stimulatory or inhibitory hematomodulators with respect to ABME formation.

### DETAILED DESCRIPTION OF INVENTION

I. Composition:

Accordingly, in one aspect, the present invention is directed to a composition useful in developing an artificial in-vitro bone marrow environment (ABME) for regulated formation of blood cells, comprising:
(i) mesenchymal cells,
(ii) hematopoietic modulators selected from biological agents, chemical agents and immunological agents; and
(iii) media for ABME creation and to practice the art.

The term ‘hematopoietic modulators’ as used herein denotes an agent that is capable of altering one or more steps of in vitro hematopoiesis, from a reference where it is not used, by which, either more blood cells in one or more lineages are formed or the relative proportion of blood cells in two or more lineages are changed, and acts through its modulatory effects on intracellular signals of mesenchymal cells or on SPC or on both. Such an agent may be a biological agent, a chemical agent or an immunological agent. Hematomodulators are essential for the development of the artificial in vitro BME and the invention provides some examples of hematomodulators. Three kinds of hematomodulators have been found to be effective; a) Biological, b) Chemical and c) immunological. These modulators are referred herein as ‘hematopoietic modulators’ or ‘hematomodulators’.

Biological Hematomodulators:

The biological agent or biological hematomodulator may be selected from 1) suitable conditioned media prepared from cells; 2) growth factors and cell stimulators such as transforming growth factor beta(TGFβ), fibroblast growth factors (FGF), vascular endothelial growth factors (VEGF), or 3) natural proteins such as fibronectin, vibronectin, laminin, collagen and their fragments containing integrin binding or activating domains and modulators of their receptors such as integrins. The said natural protein includes proteins derived from naturally occurring homologous genes across species and genera and their synthetically generated functional homologues or mimetics. Such biological hematomodulators act by generating or sustaining multiple intracellular signals in target cells salutary for SPC-homing, SPC-self renewal, SPC-engraftment, SPC-commitment to lineages for differentiation and robust blood cell formation. The concentration range of the biological agents in the composition may be about 0.1 nano-molar to 50 micro-molar.

Chemical Hematomodulators:

The chemical agent or chemical hematomodulator employed as hematomodulators may be boosters or modulators of specific intracellular signaling in target cells that are salutary to SPC-homing, SPC-self renewal, SPC-engraftment, SPC-commitment to lineages for differentiation and robust blood cell formation. Such a chemical agent may or may not be structurally related to proteins, peptides or their functional homologues and may act as boosters of specific intracellular signals. The chemical hematomodulator may be selected from:

(i) a protein kinase C booster, (ii) a booster of cyclic Guanosine monophosphate (cGMP)-activated processes including the protein kinase, (iii) a booster of focal adhesion kinase, (iv) a booster that concomitantly activates PI3-kinase, PD kinase, Akt kinase and other downstream members of Akt activation pathway, (v) a modulator of Ca⁺⁺ signaling, including Ca⁺⁺-Calmodulin-dependent protein kinase (vi) a booster of integrin linked kinase, and (vii) a combinatorial booster, comprising suitable combinations of two or more boosters selected from (i) to (vi). The peptide and protein reagents described heretofore follow and specify individual natural amino acids by their three letter codes and the sequence string is described starting with the N-terminus and ending with C-terminus.

The protein kinase C boosters may be a lipid like substance such as natural or synthetic diacyl glycerol, Farnesyl thiothriazole or a non-lipid chemical such as (−) Indolactam V, members of phorbol ester group exemplified by 12-O-tetradecanoyl phorbol 13-acetate, or agents that inhibit diacylglycerol lipase enzymes in the cell such that diacyl glycerols generated in the cells are able to function for longer periods, exemplified by 1,6-bis(Cyclohexyloximinocarbonylamino) hexane(U-57908). A booster for cGMP-activated processes may be a cGMP like compound, which may readily enter cells and boost the cGMP-dependent processes including a protein kinase directly or indirectly. Such compounds may be 8-(4-Chlorophenylthio)guanosine3′,5′-cyclic monophosphate salts, Adenosine 3′,5′-cyclic monophophothioate-Rp-isomer salts or compounds inhibiting destruction of cGMP within the cells by its specific phosphodiesterases, such as Zaprinast and Sildenafil and their functional homologues. The Focal adhesion kinase booster may be a protein or even peptide motif that is capable of interacting with mesenchymal cells or particularly with the various integrin molecules present on their cell surface consequent to which the Focal adhesion Kinase is activated and concomitantly integrin receptor-related signals within the cells are generated, enhanced or sustained. Hematomodulators of linear peptide nature are described herein and in all cases as, having the peptide/protein sequence described in standard three letter codes for amino acids and the sequences starting from amino terminal end and terminating with the carboxy terminal amino acid. Examples of peptide hematomodulators are: Trp-Gln-Pro-Pro-Arg-Ala-Arg-Ile, linear or “head to tail cyclic peptides” comprising the sequence motif “Arg-Gly-Asp-Serine” or its functional homologues; the boosters of integrin linked kinase, PI3Kinase and Akt-kinase are exemplified by the peptides, Trp-Gln-Pro-Pro-Arg-Ala-Arg-Ile, linear or cyclic peptides comprising the sequence motif “Arg-Gly-Asp-Ser”, and the protein TGFβ1.

The chemical agent may be a calcium mobilizing agent that releases Ca⁺⁺ ions from intracellular stores, or allows more external Ca⁺⁺ ions to enter cells via activation of Ca⁺⁺ channels, consequent to which several Ca⁺⁺ dependent enzymes including protein kinases are activated, such hematomodulators are exemplified by thapsigargin, cyclopiazonic acid and 8-(N,N-diethylamino)octyl-3,4,5-trimethoxybenzoate (TMB-8). The chemical hematomodulator may be an inhibitor of tyrosine kinase within the mesenchymal cells, exemplified by 3-Amino-2,4,-dicyano-5-(3′,4,5′-trihydroxyphenyl)penta-2,4-dienonitrile (Tyrphostin AG183, synonym Tyrphostin A51).

The chemical hematomodulator may be a regulator of FGF receptor function such as peptide Ala-Pro-Ser-Gly-His-Tyr-Lys-Gly, which is used on mesenchymal cells to form ABME as such or as a synergistic booster of ABME formed by another hematomodulator such as, TGFβ1.

Further, the hematomodulator may be an agent that promotes or fosters signaling through diffusible chemical messengers, such as Nitric Oxide, Stromal Cell Derived Factor-1 alpha (hereafter as “SDF-1alpha)and Stromal Cell Derived Factor-1 beta”, (hereafter as “SDF-1beta”). The inventors have found that the mesenchymal cells are capable of producing Nitric Oxide, SDF-1alpha and SDF-1 beta, and after the composition of ABME is formed, these chemical messengers are produced in enhanced amounts by the ABME. Nitric Oxide, SDF-1 alpha and SDF-1 beta are involved in the functional mechanisms of ABME to stimulate robust hematopoiesis as explained hereafter. Molecules of SDF-1 alpha and SDF-1 beta serve as attractants of SPC and allow the chemotactic navigation of SPC from long distances to reach ABME. In situations where SDF-1 alpha, SDF-1 beta secretion from mesenchymal cells are increased, the chemotactic gradients formed by them become stronger and reach longer distances effectively increasing their sphere of influence and facilitating the collection of SPC from larger volumes of environment surrounding the ABME. SDF-1alpha and SDF-1 beta help engraftment and also act as inducers of proliferation of SPC and progeny derived therefrom, thus facilitating robust hematopoiesis.

Inventors have determined that hematomodulators can increase the expression of gap junction proteins such as Connexin 43 in the mesenchyme cells forming the ABME. Connexin 43 plays an important role during natural hematopoiesis facilitating inter-cellular communications.

Inventors have determined that increased Nitric Oxide contents in ABME have a salutary effect on robust blood cell formation. Nitric oxide is highly reactive and very rapidly combines with molecular Oxygen and consequently gets destroyed. Accordingly, any agent that promotes an effective decrease of Oxygen content within the mesenchymal cells or induces hypoxia in them will foster a prolonged Nitric Oxide signaling and exert a salutary effect on the function of ABME. Hypoxic state of cells is also a facilitator for novel gene expression including the release of VEGF that promotes ABME function, mobilization and activation of endothelial cells to form new blood vessels promoting vasculogenesis and angiogenesis, which are intimately related to the blood cell formation in vivo. An environment comprising decreased Oxygen tension facilitates the expression of the CXCR4 receptors on the surface of SPC, and such SPC with increased CXCR4 molecules are better guided and attracted towards the ABME through SDF-1 mediated chemo-attraction. The Nitric Oxide in mesenchymal cells can be increased by artificial introduction of this diffusible messenger to the target cells from “Nitric Oxide Donors” exemplified by contacting them with reversible adducts of Nitric Oxide formed with several compounds such as S-Nitroso Penicillamine (SNP),2-(N,N-Dimethylamino)-diazenolate-2-oxide(DEANONOate)and the like, better ABME related properties are exhibited by the contacted cells.

The hematomodulator may be a chemical agent which is a linear or cyclic peptides comprising of motifs capable of activating integrin receptors, Focal Adhesion Kinase, bFGF-receptor of mesenchyme cells. Such a modulator of alhpa5:beta1, modulator of alpha2:beta1, modulator of alpha2b:beta3, modulator of alphaV:beta5, modulator of alphaV:beta3, modulator of alpha4:beta1, fibronectin adhesion promoting factor (FAK-activator), integrin modulators such as Arg-Gly-Asp-Ser), bFGF regulator such as Ala-Pro-Ser-Gly-His-Tyr-Lys-Gly, natural fibronectins or sub-fragments of fibronectin containing various integrin interacting domains, cell binding domain, heparin binding domain and gelatin binding domain. The amount of the said chemical agent may be about 0.1 to 100 micromolar.

The hematomodulator may be a chemical that is capable of preventing the intracellular degradation of proteins or transcription factors involving Oxygen dependent death domain motif, an example of which is HIF-1α.

Immunological-Hematomodulators:

The Immunological-hematomodulator defined herein may be an antibody or a functional homologue thereof, capable of activating cellular adhesive signals, especially from integrin receptors in the target cells such as mesenchymal cells. A selected non limiting example of an immunological hematomodulator is an activating antibody to the integrin beta subunit. Such an immunological hematomodulator may be used at a concentration that is sufficient to cause aggregation of target cells to the extent of 50% or more.

Priming Hematomodulators:

Some of the biological, chemical or immunological hematomodulators may optionally be used to contact SPC to prime or activate them prior to their use with ABME for better results. Some examples of priming hematomodulators are: A poly (ADP-ribose) polymerase inhibitor (3 amino benzamide), latency associated peptide for TGF beta 1, a soluble or cell surface associated mannose 6-phosphate containing glyco-conjugate, IGFI and IGFII effectors, boosters of cGMP signaling.

Where the agents recited above are proteins, it includes their homologues, synthetically generated or artificially engineered molecules.

The mesenchymal cells employed in the invention refer to cells obtained from the liver, bone marrow iliac crest, femur and rib or mesenchymal-stem cells. Usually, the cells chosen are such that they are not capable of forming hematopoietic colonies. Further, these cells may be of homogeneous or heterogeneous nature and may comprise of cells such as fibroblasts, macrophages, osteoblasts, endothelial and smooth-muscle cells. The growth medium referred to above is a medium suitable for culturing animal cells. The medium may be selected from Iscove's Modified Dulbecco's Medium (IMDM), Dulbecco's modified eagle medium (DMEM), Alpha-Minimum essential medium (MEM), RPMI-1640 supplemented with Fetal Bovine Serum (FBS) and optionally supplemented with methyl cellulose, erythropoietin, hematopoietic growth and differentiation factors and interleukins.

While practicing the art, the SPC amplified in an initial cycle of ABME contact, usually for short periods of 48-72 hours, can be isolated and further amplified by repeating such contacts with fresh ABME for one or more cycles to gain further benefits.

II. Kit

The present invention also provides a kit or plurality of kits useful for creating an artificial bone marrow environment (ABME) and using it for a variety of applications, including to achieve regulation of one or more of the individual steps of blood cell formation, comprising:
a) one or more hematopoietic modulator which may be a biological, a chemical or an immunological agent selected from those described in the earlier section,
b) a diluent for hematomodulator comprising Dimethyl Sulfoxide, phosphate buffer, IMDM,
c) a medium suitable for culturing mesenchymal cells e.g. Dulbecco's medium, RPMI-1640, IMDM with growth supplements as described in earlier section
d) a wash solution useful in removing used hematomodulators such as phosphate buffered saline or IMDM
e) a solution useful for harvesting the cells of ABME and/or recycling activated SPC for further use such as solutions comprising proteolytic enzymes, inhibitors and ethylenediamine tetraacetic acid(EDTA)
f) a solution of hematomodulators to prime the stem progenitor cells (such agents are the same as described in the earlier section)
g) wash solutions to remove the priming agents before using the primed stem progenitor cells such as Phosphate buffered saline or IMDM
h) a medium for in vitro blood cell formation comprising a supporting template or scaffold for ABME, cells of ABME, pro-hematopoietic growth, differentiation and survival factors, growth medium and optionally methylcellulose, serum, suitable scaffold and
i) manual of instructions.

The scaffold may comprise a substrate of two or more dimensional matrix of fibronectin, collagen or any other similar substrate.

The kit may optionally include other reagents for i) assessing the quality of a given mesenchymal cell population to form ABME, ii) quantitative screening of biological, chemical and immunological entities for their potential hematomodulatory functions, iii) preparing ABME and priming SPC for robust blood cell formation. iv) preparing ABME to alter the composition of blood cells formed in vitro, in a single or plurality of lineages, v) to induce quiescence on SPC present in a given sample. The reagent system is presented in a commercially packaged form, as a composition or admixture where the compatibility of the reagents will allow, in a test device configuration, or more typically as a test kit, i.e., a packaged combination of one or more containers, devices, or the like holding the necessary reagents, and usually including written instructions for the performance of assays.

The Specific Examples of the Priming Agents as Referred Above are Listed Below:

| Hematomodulator | Example |
|---|---|
| Biological agent 1 | HumanTransforming Growth Factor beta1 |
| Chemical agent 1 | (—) Indolactam V |
| Immunological agent | Activating antibody to human integrin beta3. |
| Biological agent 2 | HuFGF-2 |
| Biological agent-CM | Conditioned medium prepared as per method |
| | described in text. |
| Chemical agent 15 | Latency Associated peptide of human TGF beta1. |
| Chemical agent 16 | Mannose 6P motif containing proteins |
| Chemical agent | Peptides capable of activating various integrins |
| 11(a-e) | as detailed in table II. |
| Chemical inhibitor 11f | Peptide inhibiting integrin Alpha4: beta1 receptor |
| | function |
| Chemical 12 | cAMPS rp isomer |
| | inhibiting cAMP dependent processes in SPC |
| Chem agent 3 | DiBromo derivative of Ca⁺⁺ chelator BAPTA |

III Synergy:

The art so far has believed that proliferation and growth of blood cells may occur only in the bone marrow (inside a body), since the conditions for the growth of such cells are most conducive therein. And that it is difficult to generate those conditions outside a body and achieve enhanced growth of blood cells. Contrary to these assumptions, the inventors have developed a novel composition and kit that assists in developing an in-vitro environment conducive to the regulated growth and proliferation of blood cells. The said environment is developed employing a combination of suitable cells and a medium supplemented with hematopoietic modulators and optionally a support for the ABME cells. The environment so created is very much suitable for hematopoiesis and resembles a natural BME. The ABME promotes: (i) enhanced homing by stronger chemo-attraction; (ii) enhanced engraftment by promoting cell adhesion between mesenchyme cells and SPC; (iii) enhanced survival of SPC against apoptotic signals by reducing pro-apoptotic molecules such as Bad, (iv) SPC commitment along myeloid and lymphoid lineages (v) activation of quiescent SPC to foster their proliferation along both self-renewal and differentiation pathways and (vi) induction of SPC quiescence when needed.

Further, it is found that the composition of the invention is capable of forming an artificial bone marrow like environment and promoting growth of SPC and hematopoietic cells only when all the ingredients thereof are used. Mesenchymal cells, when used as such without the treatment with hematomodulators, neither sustain nor an efficient artificial bone marrow-like environment (BME) is generated. It is only a combination of the ingredients (i.e. the cells along with treatment medium comprising appropriate hematomodulators) that yields this result. Hence, the composition and kit of the invention are synergistic and are surprisingly found to develop an artificial bone marrow like environment, which result is not observed when the ingredients are used singly. Hence, the composition and kit are synergistic.

IV. Method

In one embodiment, the invention provides a method for developing an artificial bone marrow environment, comprising the steps of:

(i) obtaining mesenchyme cells and culturing them in an appropriate growth medium,

(ii) contacting the mesenchyme cells with hematopoietic modulators for a period of 20 minutes to 24 hours whereby their intracellular signaling pathways are activated and they acquire bone marrow environment like properties;

(iii) obtaining stem progenitor cells (SPC) and optionally priming the same,

(iv) contacting the mesenchyme cells as treated in step (ii) above with SPC, which are optionally contacted with hematomodulators, for the activation of intracellular signaling pathways that enable their synergistic participation with ABME to form more blood cells;

(v) contacting the primed SPC with ABME or activated mesenchyme cells in a medium for a period of 20 minutes or longer to achieve in vitro SPC-homing, SPC-engraftment, SPC-activation, SPC-self renewal, SPC-lineage commitment along lymphoid and myeloid lineages which now yield robust hematopoiesis. The method can also be used by appropriate selection of the hematomodulators to induce SPC quiescence for specific purpose.

The mesenchymal cells may comprise of cells obtainable from iliac crest, bone marrow cells harvested from adult rib or femur bones and may be maintained in a suitable culture medium such as Iscove's Modified Dulbeccos' Medium (IMDM) supplemented with 20% human or fetal bovine serum and under conditions whereby they grow and yield 10⁷ or more mesenchymal cells in about 3-6 weeks after 3-4 serial passages in routine cell culture. The “cell culture condition” comprises of maintaining the culture at 37° C. and in a humidified atmosphere of 5-7% Carbon dioxide under sterile conditions in an appropriate cell culture grade plastic ware (Becton-Dickinson, USA).

As described above, the medium for blood cell formation employed may be IMDM with 20% serum and supplemented with interleukins such as IL-lbeta, IL-3, and IL-6, stem cell factor, lineage specific growth factors such as erythropoietin and GM-CSF, G-CSF; and methyl cellulose to the extent of 0.8%, the culture is maintained for 12-14 days under the culture conditions whereby blood cell colonies grow well or form colonies and clearly the growth is visible/distinguishable for further analysis and use. An assay designed in a similar manner to assess quantitative nature of ABME function is referred hereafter as ‘quantitative hematopoietic colony formation assay for ABME (HCFA-ABME)’.

The amount of mesenchymal cells to be used for ABME creation is dependent upon the number of SPC to be processed with ABME, and the method can be suitably scaled to meet any increase or decrease in the required mesenchymal cell numbers. Usually, the amount of mesenchymal cells may be 1.5 fold of the target SPC.

The step of contacting mesenchymal cells with hematopoietic modulators may be achieved by covering mesenchymal cells with an appropriate solution/suspension of hematomodulator in IMDM with 20% serum and keeping the cells in a standard cell culture condition for periods of 0.5-24 hours whereby the cells are activated and ABME like properties are induced, which last for sufficient duration to remain useful, even after the hematomodulator application is withdrawn.

Optionally, the SPC or a population of cells that contain SPC ( Examples: Mononuclear cells isolated from the bone marrow, nucleated cells from cord blood, nucleated cells from peripheral blood after mobilization of SPC) may be mixed with the prepared ABME such that the ABME cells are nearly 1.5 times in excess compared to the SPC cells used. The SPC and ABME may be suspended in a common medium and kept together for a period of at least 30 minutes under appropriate culture conditions. When SPC are made adherent on a suitable surface ABME cells can be coated on or around them. Further, a medium comprising IMDM, 20% serum and 0.8% methylcellulose supplemented with other reagents as required by each specific application may be used to coat both the ABME and SPC.

The concentration at which a particular hematomodulator is to be used and the duration for which a given batch of mesenchymal cells is to be contacted for obtaining optimal results may vary and these exact details are to be determined in each individual case, a useful range of concentrations and duration of their treatment for hematomodulators described herein serves only as a guideline.

In another embodiment, the process is adapted to a compartmental culture capable of reducing the inhibitors of hematopoiesis generated in situ during incubation by diffusion wherein the cells of ABME and SPC are contained in one compartment and the culture medium with necessary or desirable growth and differentiation factors or hematomodulators are contacted to the cells from a separate and replaceable compartment.

In yet another embodiment, the process is adapted to a flow culture system wherein the cells of ABME and SPC are contained in a support/compartment, allowing the application media comprising culture medium, contacting medium with hematomodulator, wash solutions, differentiation medium to percolate or flow through the cells in a programmed manner for the purpose of avoiding the accumulation of hematopoietic inhibitors generated naturally in situ and allowing continual nourishing of cells to improve results.

Inventors have found during the course of their investigations that it is not obligatory to continue the contacting of mesenchymal cells with hematomodulators when ABME are found to be ready or SPC are found to be activated for promoting robust hematopoiesis. When the same numbers of SPC are contacted with mesenchymal cells as reference or with ABME under otherwise comparable experimental conditions, a significantly high number of SPC are recruited to ABME and when they are further processed for HCFA a significant increase in colony number is found in ABME compared to the reference implying enhanced homing, more effective engraftment and robust blood cell formation. This result signifies an increase in the proportion of the activated SPC present in the sample implying further that some SPC might have exited the quiescent state and entered into an activated state and/or may have undergone self renewal to increase the proportion of activated SPC cells.

In another embodiment, the inventors have identified negative hematomodulator which modulate the functionality of ABME such that the normal SPC attain the equivalent of quiescent state. Such a modulation allows development of protocols to distinguish between normal and pathological SPC having differences in their cell cycle regulation characteristics. Thus the process supports a therapeutic regimen for the selective destruction of leukemic SPC in vitro using anti-neoplastic drugs avoiding the adverse side effects associated with chemotherapy in vivo.

The invention is now illustrated by the following examples that are provided for illustration only and not meant to limit the scope of the invention or inventive concept.

### EXAMPLES

### Example 1

a) Obtaining and Preparing Mesenchymal Cells:

Bone marrow cells obtained from iliac crest or rib bones, are dispersed well in Iscove's Modified Dulbecco Medium (IMDM) to obtain a single cell suspension and are washed at least three times with the same medium by collecting cells each time by centrifugation in a centrifuge at 2-3000 RPM (500-1000 Xg) for 5 minutes. The washed marrow cells (at least 10⁸ cells) are kept up to 7 days in IMDM and 20% fetal calf or human serum under cell culture conditions. The adherent cell layer on a typical standard tissue culture grade plastic ware (T-25 flasks, Becton Dickinson, USA) thus obtained in a few days, is washed with plain IMDM and expanded further in several passages of standard cell culture in the same medium. Typically, after 3 passages the intrinsic hematopoiesis i.e. its ability to give rise to hematopoietic colonies in methylcellulose assays is lost. At least 10⁷ mesenchymal cells are obtained starting from 10⁸ bone marrow cells after 3-6 passages. The mesenchymal cells obtained this way are suitable for the generation of ABME by methods described herebelow.

b) Preparation of SPC Suitable for use with ABME.

The washed marrow cells obtained as in step (a) above are carefully layered on a gradient of Ficoll-Hypaque (density 1.007, Sigma Chemical Company, St. Louis, USA) and centrifuged at 1500 RPM (1000 Xg) in a swing out rotor of Kubota centrifuge (Kubota Corporation, Bunkyo-ku, Tokyo, 113-0033, Japan) for 15 minutes. The mononuclear cells (MNC) are collected from the interface layer and are washed 3 times with IMDM by consecutive centrifugation at 1000 Xg and resuspensions. The MNC are then suspended at a suitable density (10⁵-10⁷ MNC/ml] in IMDM supplemented with 20% human or fetal bovine serum. MNC prepared here may be used as such with ABME. The SPC are likely to be found in vivo in an environment similar to MNC isolated here.

For illustrative purposes, the method has been tested both with MNC as well as with highly enriched, CD34⁺ antigen expressing SPC to simulate the above two conditions. When required, CD34⁺ cells were recovered routinely from MNC fraction, by using a CD34⁺-cell isolation kit (Dynal, Smestad, N-0309, Oslo, Norway), as per the manufacturer's instructions, although any other suitable method can be used. CD34⁺ cells may also be harvested by leukapheresis after their mobilization from the marrow by a suitable method or from cord blood. CD34⁺ cells formed from their CD34⁻ (CD34 negative) precursors before or after their presentation to ABME also yield desired results. The SPC prepared in this manner also remain suitable for the priming with suitable Hemato-modulators.

c) Mesenchymal Cells Stimulate Hematopoiesis from a Fixed Number of SPC:

Several experiments were conducted to determine that mesenchymal cells exert direct influence on SPC cell-fate. In one set of experiments, mesenchymal cells were cultured separately and nearly 50,000 of them were exposed to a typical HCFA medium comprising 0.8% methyl cellulose in IMDM and 20-30% serum along with excess amounts of various purified and human specific cytokines and hematopoietic colony stimulating growth factors (from Stem cell Technologies, Toronto, Canada). More specifically, the quantities of hematopoietic growth factors used routinely in HCFA were, 2 International Units per milliliter (2 I.U.ml⁻¹) of Erythropietin (EPO), 50 nanogram per milliliter (50 ng.ml⁻¹) of Stem Cell Factor (SCF) and 20 nanogram per milliliter (20 ng/ml⁻¹) each of Granulocyte-Macrophage-Colony Stimulating Factor (GM-CSF), Granulocyte-Colony Stimulating Factor (G-CSF), Interleukin-1 beta (IL-1β), Interleukin-3 (IL-3) and Interleukin-6 (IL-6) {all growth factors and cytokines are from Stem cell Technologies, Vancouver, Canada,}. It was found that the mesenchymal cells prepared by the method described herein, were unable to form hematopoietic colonies on their own in the colony formation assay medium even though excess amounts of all required growth factors were provided.

In another set of experiments, 50,000 of the same mesenchymal cells were first mixed with nearly 100,000 MNC, held at 37° C. for at least 30 minutes and were processed for colony formation assays. In several experiments, a consistent improvement in the number and cellularity of the hematopoietic colonies was seen when both these two cell types were mixed compared to a reference where only the cells of MNC were used. The increase was ranging from 1.5 to 10-fold. Thus, the enhancement in colony formation obtained with the use of only mesenchymal cells was highly variable and unpredictable among samples. The fact that hematopoietic colonies formed in increased numbers and cellularity only when MNC and mesenchymal cells were processed together is evidence of a new environment created by the mesenchymal cells in which the SPC present in the MNC were more productive. This increase results in activation of the pre-existing but quiescent SPC, formation of new SPC due to self-renewal, and a combination of both factors.

d) Contacting Hematomodulator on Mesenchymal Cells Yields Better Results:

The practical use of the hematomodulators needs optimization with regard to their dose and the duration of contacting to be employed, since different batches of mesenchymal cells may respond to the same hematomodulators differently. From many experiments, the inventors determined that in general, a convenient starting point could be: the use of Biological-hematomodulators in the range of 1-25 nanograms/ml(with respect to their active ingredients), Chemical hematomodulators in the range of 10 nMolar to 500 microMolar solutions and Immunological hematomodulators in the range of 10-50 pico Molar solutions.

In order to create ABME, the mesenchymal cells were covered with the contacting medium comprising hematomodulators contained in IMDM with 20% human serum or fetal bovine serum for a desirable period (range 8-24 hours) and removed. The contacted cells were then washed 2 times using the contacting medium (IMDM +20% FCS) without hematomodulators and used as such or after harvesting the mesenchymal cells, which now represent the composition of in vitro ABME. Optionally, ABME cells can be configured on supports that will distribute them in two or more dimensions for better results. A requisite number of MNC/SPC and cells of the ABME are taken and kept together for a suitable duration (range 0.5 to 6 hours) after which they were processed for in vitro hematopoietic colony formation assays or hematopoiesis.

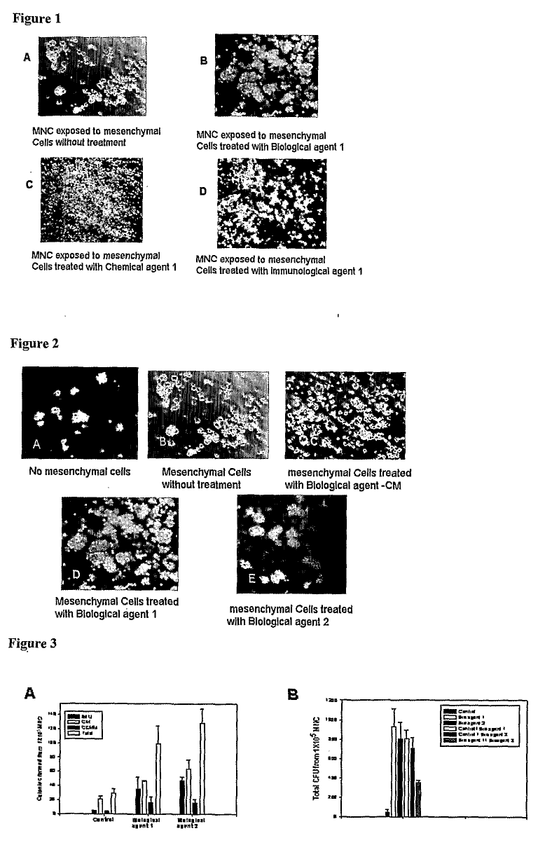

Thus, the inventors undertook 4 sets of experiments: (a) wherein mononuclear cells were exposed to mesenchymal cells without any treatment; (b) wherein MNC were exposed to mesenchymal cells contacted with a biological agent [TGFβ1] (c) MNC exposed to mesenchymal cells contacted with a chemical agent 1 [12-o-tetradecanoyl phorbol,13 acetate/(−)Indolactam V give name of agent here] and (d) MNC exposed to mesenchymal cells treated with an immunological agent [activating, anti integrin beta 3 antibody]. The results are shown in FIG. 1 and are as under:

(A) In this situation, few blood cell colonies of low cellularity were formed;

(B) Blood cell colonies were formed which were greater in number and of higher cellularity than (A);

(C) Large colonies of cells were observed, surprisingly at least 4 times greater than (A) and greater than (B);

(D) Dense cellular colonies were formed which is surprisingly greater than (A) or (B).

### Example 2

### Effect of Biological Hematopoietic Modulators

The preparation method comprises: MNC (>10⁷ cells) held in one milliliter of a medium comprising IMDM, 20% serum and a suitable modulator (Example Erythropoietin and GM-CSF) that is/are necessary in appropriate amounts (Example: Erythropoietin 2 I.U.ml⁻¹; GM-CSF 20-50 ng.ml⁻¹) to release the hematomodulator-CM from the cells under cell culture conditions at 37° C. After a suitable period (range 8-96 hours), the cells are removed by centrifugation in a refrigerated Kubota centrifuge (5000 Xg, 15 minutes) and the Biological hematomodulator-CM obtained as the supernatant, is used as such, or it is used after further processing to concentrate the active ingredients present. The processing of CM generally took place in a chilled environment of ˜4° C. and made use of the principle of affinity chromatography. Briefly, a suitable affinity ligand immobilized on a matrix (Example Heparin Sepharose) was taken, the hematomodulator-CM was absorbed to it in a low salt buffer, unabsorbed components were washed away by the loading buffer, and active ingredients of hematomodulator-CM was first selectively eluted by a high salt (Example 1.5 Molar NaCl) buffer, and then concentrated and equilibrated to the storage buffer, preserved at −70° C. for future use. The other two biological hematomodulators, namely, Biological agent-1 and Biological agent-2 were identified to be TGFβ1 and FGF-2 respectively.

Biological hematomodulator-CM, TGFβ1 and FGF-2 create equally efficient ABME under appropriate conditions; yet, the nature of the ABME formed in each case is distinct. Typically, the Biological hematomodulator-CM, TGF-β1 and FGF-2 are used in the concentration ranges of 1-25 nano gram.ml⁻¹ in IMDM and 20% human or fetal bovine serum to contact the mesenchymal cells for 8-24 hours under cell culture conditions to generate the ABME. As shown in FIG. 2, stronger ABME related properties become manifest in the culture only when the hematomodulators are used (FIGS. 2C, 2D and 2E) but not, when mesenchymal cells are not used (FIG. 2A) or if used without Biological hematomodulators (FIG. 2B). The figures show the density of blood cells formed in each case, which relates to the cellularity of the colonies. The best colonies are formed only when mesenchymal cells are contacted with hematopoietic modulators.

### Example 3

### Effect of Biological Hematopoietic Modulator on Colony Formation

The inventors have determined by experiments that ABME obtained by the use of TGFβ1 and FGF-2 are nearly equipotent in creating the ABME from mesenchymal cells when used separately and each such ABME is freely miscible without significant attenuation with its parental mesenchymal cells.

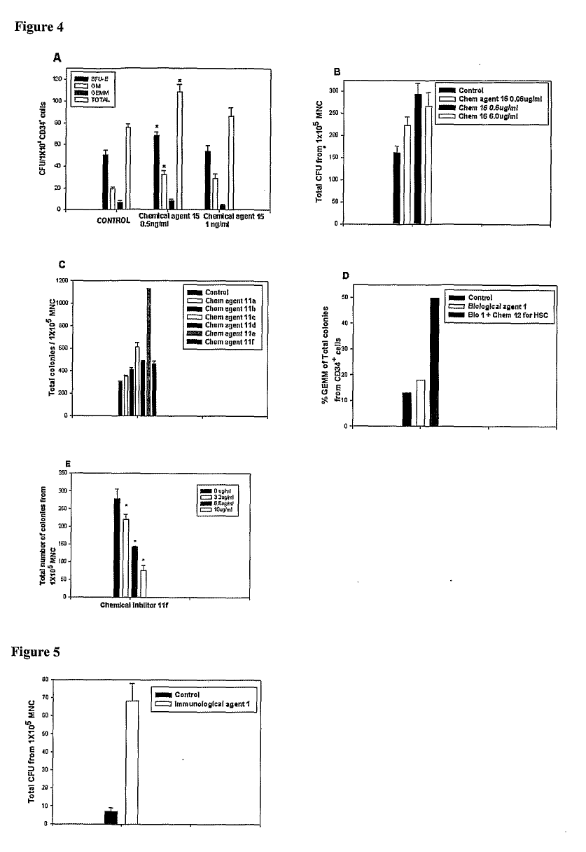

A set of experiments pertaining to quantitative, lineage specific colony formation assay using mesenchymal cells was performed: (A) without contacting with any Biological agent [FIG. 3A control] or after their contact with TGFβ1 [FIG. 3A Biological agent 1] or FGF-2 [FIG. 3A Biological agent 2]. It is evident that the colony formation along several specific lineages including Granulocyte-Erythroid-Monocyte and Macrophage (GEMM), Burst Forming Unit-Erythroid (BFU-E) and Granulocyte-Macrophage (GM) are uniformly stimulated by both the ABME and that TGFβ1 and FGF-2 were nearly equipotent in creating the respective ABME which sustained this effect. In particular, stimulation of GEMM colony formation is indicative that the ABME is capable of stimulating multipotent progenitors to form more new multipotent cells equivalent to self-renewal.

In FIG. 3B, applicants have shown that ABME created by the use of TGFβ1 or FGF-2 is both compatible with the untreated mesenchymal cells when tested individually. Each of these two ABME is therefore suitable for mixing with the untreated mesenchymal cells. By contrast, when both the ABME are mixed in equal proportions, the combination is a weaker ABME than the individual one. This clearly proves that they are mutually antagonistic in nature and are non-identical.

### Example 4

### Effect of Chemical Hematopoietic Modulators on Colony Formation

The inventors have tested several hematopoietic modulators that modulate intracellular signal generation and/or sustenance such as functions of a variety of protein kinases, particularly that of, cGMP dependent kinases, lipid-dependent kinases (Example: PKC), phosphatidyl inositol phosphate dependent kinases and family (PI3K, PDK, Akt, pbad, mTOR), cell-adhesion dependent kinases(FAK,ILK), receptor tyrosine kinases (Example: FGFR, VEGFR, IGF-1R, IGF-2R), receptor-Serine/Threonine kinases(Example: TGFβ1 Receptors) and intracellular Ser/Thr kinases(Example: MAPK-kinase and p38 MAPK kinase), Ca⁺⁺ ion dependent kinases(Ca⁺⁺-CaM dependent kinase), signifying that any new molecule capable of bringing about such functions is a potential hematomodulator by implication.

For ABME creation, mesenchymal cells were contacted for a suitable duration (Range 5 minutes-24 hours) using IMDM with 20% Human or Fetal calf serum where an appropriate amount of hematomodulator was also present. Preparation of effective hematomodulator solutions and duration of contacting mesenchymal cells, require careful optimization since neither a higher concentration of the hematomodulator nor a longer contacting time guarantee better results; a useful range being 1 nMolar to 100 μMolar for hematomodulators and 5 minutes to 24 hours for duration of contact.

In another aspect, the inventors have determined that some combinations of a biological agent and a chemical agent may act synergistically and lead to a better ABME creation compared to that when any one of them is used.

In another aspect, the inventors have determined that suitable chemical hematomodulators can be used to suppress blood cell formation. These are described herein as “negative hematomodulators” to indicate that they promote SPC quiescence. Such hematomodulator, if it is dominant in the context, can attenuate the stimulatory action of another hematomodulator.

FIG. 4A shows that the use of different hematopoietic modulators creates ABME that is capable of stimulating blood cell formation differentially in various lineages. FIGS. 4B and 4C shows the effect of Chemical hematopoietic modulators on colony formation. FIG. 4D shows the effect of combination of biological and chemical hematopoietic modulator is capable of showing synergy in forming ABME. FIG. 4E shows the dose dependent attenuation of an ABME by a negative hematomodulator.

### Example 5

### Immunological Hematomodulators

The inventors have determined that an antibody reagent or its functional homologues capable of activating adhesive interactions on mesenchymal cells through the integrin receptors act as hematomodulators. Further, such immunological hematomodulators may act synergistically with Biological hematomodulators and/or Chemical hematomodulators to create better ABME in vitro. Such immunological hematomodulators are useful in the range of 10-100 microgram.ml⁻¹ in a medium comprising IMDM with 20% human or fetal bovine serum. The mesenchymal cells are contacted with this medium for a suitable period (range 1-24 hours) under culture conditions by which mesenchymal cells form ABME. In another aspect, the use of immunological hematomodulator can be combined with the use of Chemical and/or Biological hematomodulators for obtaining better results.

As shown in FIG. 5, only a few blood cell colonies are formed when the mesenchymal cells are used (Control: Dark Bar). By contrast, when a solution of Immunological hematomodulator,( antibody to human beta3 integrin subunit), at 50 micro gram per milliliter suspended in IMDM with 20% human or fetal bovine serum) was used to contact mesenchymal cells for 1 hour, the mesenchymal cells formed a highly effective ABME resulting in an enhancement of colonies and blood cell formation in vitro.

### Example 6

### Improving Results Further by Priming SPC Before Contacting them with ABME

The SPC or a population of MNC having SPC in them can also be separately primed with specific chemical agents so that they will form more and better blood cell colonies after contacting them with ABME. The priming of SPC may itself form better and more colonies compared to cells without priming but best results are obtained when the primed SPC are combined with ABME. The inventors have identified at least ten different chemicals capable of priming SPC and leading to improved colony/blood cell formation signifying that this methodology is also useful for the identification of new priming agents (latency associated peptide, 3-aminobenzamide, IGF-1 & II, mannose 6 p containing glycol conjugates). The priming agents were capable of activating signals related to integrin mediated adhesion, IGF-I receptor, Mannose6-phosphate/IGF-II receptor, cGMP dependent functions, or inhibiting the Poly(ADP-ribose phosphate) Polymerase function on SPC signifying that any new molecules functionally homologous to these can be accepted as priming agents. Since these priming agents modulated hematopoiesis significantly, they are also accepted as hematomodulators.

### Example 7

### Modulation of Homing

Experiment A

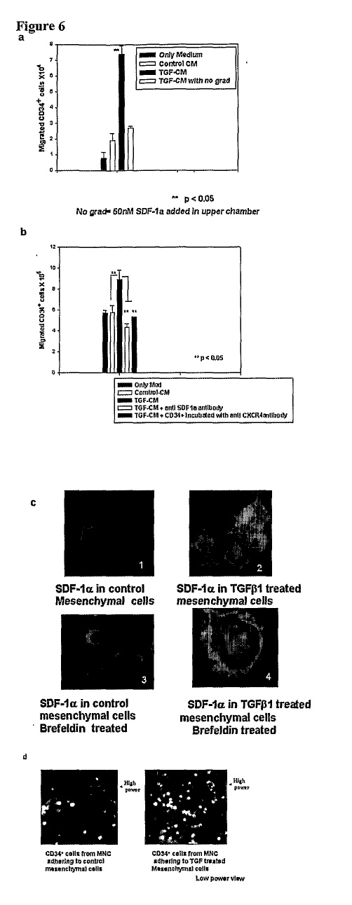

5×10⁵ mesenchyme cells were grown on a 35 mm diameter Petri dish (Becton Dickinson, USA) in IMDM containing 20% serum. After the cells were confluent, the medium was removed, the cells were washed 2× with Phosphate buffered saline (GIBCO-BRL) (PBS) and the medium was changed to treatment medium containing 10 ngml⁻¹ of TGFβ1 and the dishes were returned to standard cell culture environment at 37° C. in 5% CO₂. After 4 hours, the treatment medium was removed, the cells were washed with PBS and were covered with 1 ml of IMDM containing 0.5% serum and incubation at 37° C. in 5% CO₂ was continued. After 18 hours, the conditioned medium was collected and was assayed for its ability to support in vitro homing of CD34⁺ SPC. In another parallel experiment, an equal number of mesenchymal cells were employed, where TGF-β1 was not used and the conditioned medium obtained from this experiment was used as a reference for comparison. The results are shown in FIGS. 6(a-c). It was seen that after the treatment of mesenchymal cells with TGFβ1, whereby they form an active ABME, significantly more amounts of SDF-1α was released which is capable of setting up a chemotactic gradient in the vicinity of ABME for attracting the SPC towards it, thus promoting their homing. As shown in FIGS. 6(a-b) untreated mesenchymal cells show no or marginal homing as compared to mesenchymal cells treated with TGF-β1. The homing could be modulated in this case either by adding suitable amounts of a neutralizing antibody to SDF-1α or to its receptor CXCR-4 to neutralize its effects on chemo-attraction of SPC or by destroying the gradient by exogenous addition of SDF1α indicating the specificity of the process.

Experiment B

Mesenchyme cells were grown on a sterile 1 cm×1 cm glass coverslip to near confluence and were covered with a treatment medium containing 10 ngml⁻¹ TGF-β1 and were incubated for 18 hours in a humidified sterile atmosphere and 5% CO₂. The coverslips were then washed with PBS, and the ABME thus created were covered with a suspension of 2×10⁶ MNC or 2×10⁵ CD34⁺ cells in 0.2 ml of IMDM with 20% serum. After 1 hour at 37° C., the coverslips were washed with PBS and the cells were fixed and stained with a CD34⁺ specific antibody (clone HPCA1, Becton Dickinson, USA). The results are shown in FIGS. 6(d-f). The number of CD34⁺ cells attached were counted and compared to the number of similar cells that were attached in a parallel reference experiment where TGF-β1 was not used. It was seen that the ABME formed by TGFβ1 on mesenchyme cells had significantly more number of CD34⁺ cells attached in comparison to the reference coverslip indicating that homing of these cells to ABME was superior to the mesenchyme cells alone. It may be seen from FIGS. 6(d-f) that there is an increased adhesion of CD34⁺ cells to mesenchymal cells when mesenchymal cells are treated with TGF-β1.

### Example 8

### Modulation of Engraftment

Engraftment experiments were directed to determine if the adhesion of the CD34⁺ cells to ABME seen in drawing 6d was functionally relevant and if so what mechanism was being used by the ABME to promote such engraftment.

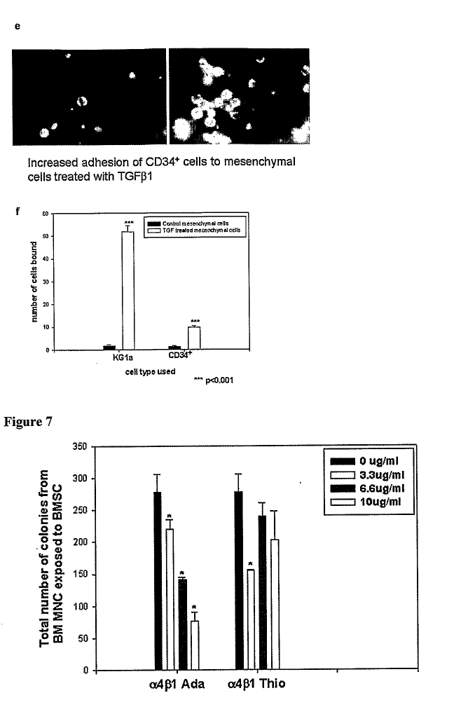

The mesenchyme cells (5×10⁴ cells per well in a 24 well dish) were grown in IMDM plus 20% fetal bovine serum until they were nearly confluent. The medium was removed and was washed 2× with PBS. ABME was prepared by using a chemical hematomodulator [Biotin-Ser-Gly-Ser-Gly-Cys*-Asn-Pro-Arg-Gly-Asp (Tyr-OMe)Arg-Cys*Lys (cyclised between C*-C*), 10 microgram ml⁻¹, 18 hours] that selectively activated αiib:β3 integrin signaling in the mesenchyme cells. The ABME formed was washed 2× with PBS and was covered with 0.2 ml of a suspension of CD34⁺ SPC cells (10⁵ ml−1) for 1 hour, in the presence or absence of another peptide [1-Adamantaneacetyl-Cys*Gly-Arg-Gly-Asp-Ser-Pro-C(Cyclised between Cys*-Cys*)]which inhibited the interactions of αiib:β3 integrin. At the end of incubation, the cells were washed 2× with PBS and were processed for hematopoietic colony formation assays (HCFA). For the hematopoietic colony formation, the ABME cells with the attached SPC were collected from the multi-well dish, were resuspended in 1 ml of IMDM containing 20% serum, 0.8% methyl cellulose and excess amounts of various purified and human specific cytokines and hematopoietic colony stimulating growth factors (from Stem cell Technologies, Vancouver, Ontario, Canada). More specifically, the quantities of hematopoietic growth factors used routinely in HCFA were, 2 International Units per milliliter (2 I.U.ml⁻¹) of Erythropoietin (EPO), 50 nanogram per milliliter (50 ng.ml⁻¹) of Stem Cell Factor (SCF) and 20 nanogram per milliliter (20 ng/ml⁻¹) each of Granulocyte-Macrophage-Colony Stimulating Factor (GM-CSF), Granulocyte-Colony Stimulating Factor (G-CSF), Interleukin-1 beta (IL-1β), Interleukin-3 (IL-3) and Interleukin-6 (IL-6). The cells were incubated for 12-14 days until the hematopoietic colonies were large. The results are shown in FIG. 7. It was seen that ABME formed by the peptide chemical modulator was capable of forming significantly more number of hematopoietic colonies whereas when this hematomodulator function was inhibited by the inhibitory peptide, the colony numbers decreased in an inhibitor dose dependent manner. The results clearly show that the contacts established by the SPC to the ABME through αiib:β3 integrin interactions are functionally relevant and when impaired compromise the function of ABME.

These results therefore show clearly that the promotion of adhesion of SPC to ABME by hematomodulator is functionally relevant and is equivalent to SPC engraftment in vitro, furthermore, this engraftment can be modulated in vitro.

### Example 9

### Modulation of Lineage Commitment

Lineage commitment of SPC during hematopoiesis can affect the composition of mature blood cells formed. Lineage commitment experiments were directed to show that it is possible to alter the composition of mature blood cell formation in two lineages by the appropriate use of ABME.

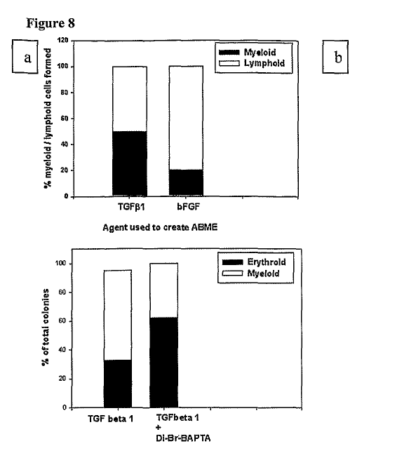

The mesenchyme cells (5×10⁴ cells per well in a 24 well dish) were grown in IMDM plus 20% fetal bovine serum until they were nearly confluent. The medium was removed and was washed 2× with PBS. Two different ABME were prepared by using two biological hematomodulators (TGFβ1 10 ngml⁻¹, bFGF 10 ngml⁻¹, 18 hours) separately. The ABME formed was washed 2× with PBS and was covered with 0.2 ml of a suspension of CD34⁺ SPC cells (10⁵ ml−1) for 1 hour at 37° C. All the cells of the dish were then harvested after washing and were used in a HCFA. After 14 days, the mature blood cells formed were harvested and an aliquot of the suspension was examined under the microscope to identify and count the mature lymphoid and myeloid cells. The results are shown in FIG. 8a. It was seen that when ABME prepared by bFGF was used, more of lymphoid cells were produced and when TGFβ1 was used for preparing ABME, it supported more myeloid cell formation. These results show convincingly that ABME prepared by choosing specific hematomodulators can be used to modulate the lineage commitment and alter the composition or proportion of myeloid and lymphoid cells in mature blood cells formed.

This experiment was directed to show that the composition of mature blood cells formed can be altered with respect to erythroid and myeloid cells. The mesenchyme cells (5×10⁴ cells per well in a 24 well dish) were grown in IMDM plus 20% fetal bovine serum until they were nearly confluent. The medium was removed and was washed 2× with PBS. The cells in one well were treated with a DiBromo derivative of the Calcium ion chelator BAPTA (5 micromolar) for one hour whereas another well received only the medium without the BAPTA derivative. After 1 hour of incubation at 37° C., TGFβ1 was added to both the wells (10 ng ml−1) and the incubation was continued for a further period of 18 hours. The ABME formed in both wells were washed and were used for HCFA using CD34⁺ SPC. After 14 days the mature blood cells were harvested and were quantitated for the cells of erythroid and myeloid lineage. The results are shown in FIG. 8b. The results showed that when DiBromo BAPTA derivative was used for the formation of ABME, there was a significant difference in the proportion of myeloid and erythroid cell population compared to the reference experiment where DiBromo BAPTA was not used.

### Example 10

### Modulation of Cell Survival

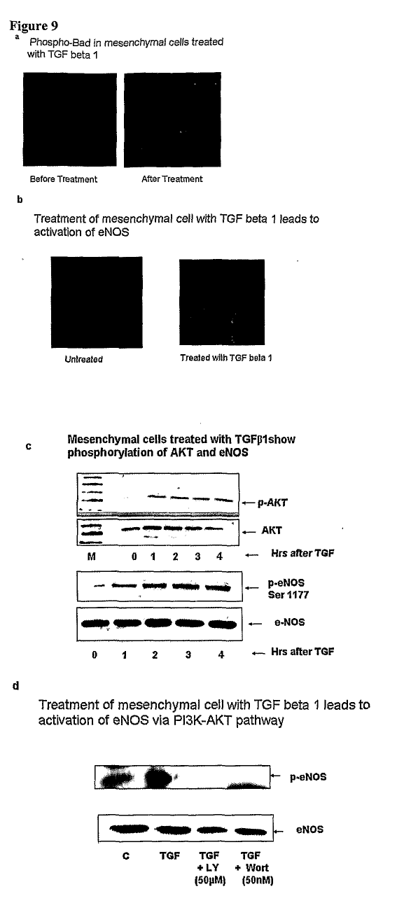

Experiments were directed to determine if the ABME had properties that promoted cell survival. When mesenchyine cells were examined for the presence of pro-survival molecules such as phospho (serine473) Akt/PKB, phosphor-Bad, it was found that these entities are present in very small quantities in the cells. However, when mesenchyme cells were used to prepare ABME, it was found that there was a significant upregulation of pro-survival factors such as phosphor(serine 473) Akt/PKB and phospho-Bad, activated Nitric Oxide Synthase in the cells of ABME. These results show that pro-survival factors are activated in ABME and therefore they can be transferred to SPC during their contacts with ABME. In another set of experiments, pro-survival signals were upregulated in SPC by the inhibition of Poly(ADP-ribose) Polymerase by using 3 Amino Benzamide. The results are shown in FIGS. 9(a-d). The results showed that after this treatment, the SPC were capable of synergistically increasing the number of hematopoietic colonies formed indicating that SPC-cell survival can be modulated in vitro.

### Example 11

### Modulation of Self Renewal

Experiment A

Experiments were directed to examine whether quiescent primitive progenitors present in MNC get stimulated when contacted with ABME thereby forming more colonies consisting of mixed lineages (GEMM).

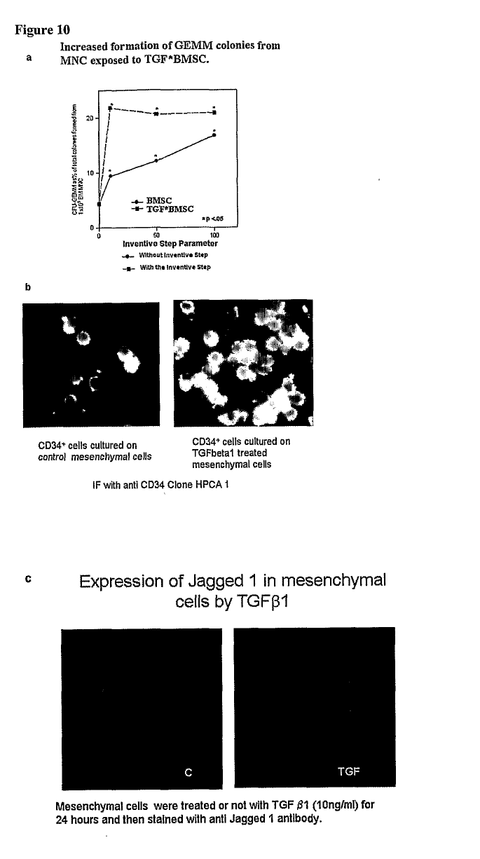

The mesenchyme cells were grown in IMDM plus 20% fetal bovine serum until they were nearly confluent. The medium was removed and was washed 2× with PBS. ABME was prepared by using a biological hematomodulator namely, TGF beta 1(20 ng ml−1).The ABME formed was washed 2× with PBS and the cells were dissociated with a non-enzymatic solution (Sigma). A fixed number of MNC namely 2×10⁵ were mixed with various doses of dissociated mesenchymal cells (2×10⁴, 5×10⁴ and 1×10⁵) and incubated for 1 hour. At the end of the incubation, cells were processed for hematopoietic colony formation assays (HCFA). A clear dose dependent increase in GEMM colony formation was observed (FIG. 10a) when the cells of ABME were used in the experiments as against control mesenchymal cells. Secondly the lowest concentration of cells from ABME (2×10⁴) was more efficient in the stimulation of GEMM formation than the highest concentration (1×10⁵) of mesenchymal cells indicating a nearly 10 fold increase in the efficiency.

Experiment B

Experiments were directed to examine if ABME offers advantage in respect of SPC-self renewal.

Mesenchyme cells were grown on a sterile 1 cm×1 cm glass coverslip to near confluence and were covered with a treatment medium containing 10 ngml⁻¹ TGF-β1 and were incubated for 18 hours in a humidified sterile atmosphere and 5% CO₂. The coverslips were then washed with PBS, and the ABME thus created were covered with a suspension of 2×10⁵ CD34⁺ cells in 0.2 ml of IMDM with 20% serum. The cells were washed after one hour and the bound cells were covered with medium with 20% serum containing 0.8% methyl cellulose and growth factors. The change in CD34⁺ cell number after 48 hours was monitored with a CD34⁺ specific antibody (clone HPCA1, Becton Dickinson, USA). The results are shown in FIG. 10b. It was found that ABME supported more CD34⁺ cell proliferation in reference to another experiment where TGFβ1 was not used. Furthermore, it was determined that the ABME contained significantly more amounts of the promoter of self renewal, Jagged1 compared to mesenchyme cells (FIG. 10c).

### Example 12

### Induction of Hypoxia Under Normoxic Conditions

These experiments were directed to examine the possibility of creating hypoxia under normoxic conditions using specific hematomodulators.

Experiment A

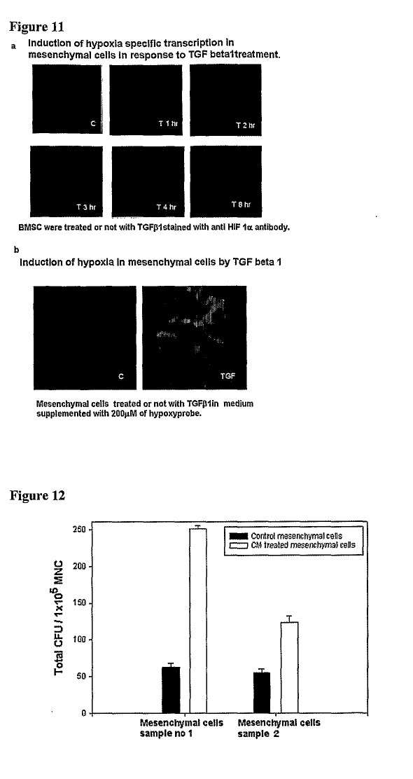

Mesenchymal cells were processed for ABME formation as per details given in experiment #1 above using a biological modulator namely TGFbeta1. The cells were fixed after the indicated time periods and immuno-stained with an antibody to HIF1α which is a specific transcription factor related to hypoxia. A clear nuclear expression of HIF1α was found in mesenchymal cells treated with TGF beta 1 as against the control cells (FIG. 11a).

Experiment B

Mesenchymal cells were processed for ABME formation as per details given in Example 7(a) above using a biological modulator namely TGFbeta1 and were incubated with 200 μM Hypoxy probe (Chemicon, USA) for 48 hours. The cells were fixed and immuno-stained with an antibody specific for detection of the label (Chemicon). The results are shown in FIG. 11b. It was observed that the ABME created by the use of TGF beta 1 showed as high incorporation of the label indicating the presence of hypoxic condition.

### Example 13

### Evaluation of Mesenchymal Cells for Suitability in Hematopoietic Functions

In the manner described in Example 8, screening of various mesenchyme cells to evaluate their potential utility in ABME formation can be performed. The results are shown in FIG. 12, wherein the efficacy of hematomodulator when contacted with mesenchymal populations cultured from two separate marrow samples is compared. Using the same batch of SPC and keeping one known hematomodulator as a reference point, one can thus evaluate the “ABME formation capacity” of any given mesenchymal cell sample.

### Example 14

### Screening for Hematomodulators

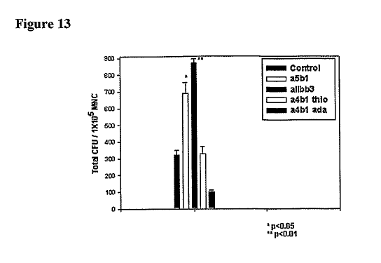

In the manner described in Example 8 novel hematomodulators may be screened by using mesenchyme cells but using treatment media having supplements of various potential hematomodulators. The results are shown in FIG. 13 wherein differential effect of a stimulatory vs. inhibitory hematomodulators with respect to ABME formation is depicted. If a known hematomodulator is incorporated as a reference point and if the same batch of SPC is used then the test substances can be evaluated for their ability to induce ABME in terms of the reference hematomodulator and can be labeled as a positive/negative; potent/less potent/ineffective hematomodulator etc.

Materials used in Invention:

All the material used in the invention(s) are available commercially. Biological material like bone marrow cells, SPC, mesenchymal cells, culture media, growth and differentiation factors, Interleukins, serum, antibodies, were obtained from biotech companies like Cambrex Bioscience, USA; American Type Culture Collections, USA; GIBCO-BRL, USA; Sigma Chemical Company, USA; Santacruz Biotechnology, USA; Neomarker, USA; Becton Dickinson, USA; Stem Cell Technologies, Vancouver, Canada; Bachem, Switzerland; Alexis Corporation, Switzerland; Promega Corporation, USA; Peprotech, U.K.; and the like. Cell separation products were from Dynal, Norway; Methyl cellulose was from Sigma Chemical Company, USA; Cell-culture related plastic ware were from Becton Dickinson, USA; Corning, Costar, USA; Cell culture related equipments such as carbon dioxide incubators were from Forma, USA; Optical equipments such as various types of microscopes were from Zeiss, Germany; Olympus Corporation, Japan and Nikon, Japan.

### ADVANTAGES AND INDUSTRIAL APPLICATION OF THE INVENTION

The composition of the present invention may be used for the creation of ABME which will be useful to:
a) modulate distinct steps of natural or artificial hematopoiesis,
b) to evaluate the bone marrow function in healthy and diseased bone marrow cells,
c) to rapidly augment natural hematopoiesis without affecting the levels of endogenous cytokines and growth /differentiation factors in the body,
d) to minimize or eliminate Graft vs Host disease observed in SPC transplantation by stimulating autologous hematopoiesis,
e) to use in vitro engrafted SPC in engineering novel functions by introducing suitable genetic or molecular entities,
f) to selectively destroy in vitro engraftable and non-engraftable SPC,
g) to promote robust growth of blood cells in one or more lineages in vitro,
h) to purge leukemia progenitor cells from the marrow by methods known in the art.
i) to promote hypoxic state in cells under normoxic conditions,
j) to induce quiescence in normal and pathological SPCs
k) to discover new drugs that will regulate one or more steps of hematopoiesis,
l) to discover new drugs that will enable the differentiation of SPC to non-hematopoietic cell fates or vice versa
m) to train newly formed immune cells to destroy selected biological targets by cell-mediated or antibody-mediated immune reactions.
n) to train newly formed immune cells to spare normal target cells of the self to prevent the debilitating effects of auto immune disorders,
o) to train newly formed immune cells to accept allogenic tissue implants by inducing tolerance.

Having now fully described this invention, it will be appreciated by those skilled in the art that the same can be performed within a wide range of equivalent parameters, concentrations, and conditions without departing from the spirit and scope of the invention and without undue experimentation.

Having now fully described this invention, it will be appreciated by those skilled in the art that the same can be performed within a wide range of equivalent parameters, concentrations, and conditions without departing from the spirit and scope of the invention and without undue experimentation.

1. A composition useful in developing an artificial bone marrow like environment (ABME) for modulating several steps of SPC-functions and bone marrow processes, comprising:
a] a culture of mesenchymal cells obtained from a mammalian foetus of human origin;
b] a hematopoietic modulator or plurality of modulators capable of activating intracellular signaling; wherein the hematopoietic modulator(s) such as herein described selected from any of: a biological agent, a chemical agent, an immunological agent and one or more suitable combinations thereof;
c] a medium for culture of mammalian cells selected from Iscove's Modified Dulbecco's Medium (IMDM), Dulbecco's modified eagle medium (DMEM), Alpha-Minimum essential medium (α-MEM), RPMI-1640 supplemented with Serum or serum substitutes and optionally supplemented with hematomodulators that promote hypoxic state in target cells, methyl cellulose, erythropoietin, hematopoietic growth and differentiation factors, Interleukin 1 beta, Interleukin 3 and Interleukin 6;
d] a support for the cells comprising constituents of extra cellular matrix or their mimetics capable of forming a matrix in two or more dimensions.

2. A composition as claimed in claim 1 wherein the concentration of hematomodulators, fetal bovine serum or serum derived from a mammalian source (5-30%), or a suitable serum substitute, erythropoietin or its mimetics: 2 IU/ml, purified growth and differentiation factors and interleukins used in the concentration ranges of 1-10 nanomolar and 0.8% methyl cellulose.

3. A composition as claimed in claim 2 wherein the hematopoietic modulator is selected from the modulators set out in Table I below.
TABLE I

| | | |
|---|---|---|
| | | Preferably Used in |
| Hematopoietic | | the Concentration |
| modulator types | Selected from but not limited to: | ranges of: |

A. Biological Hematomodulators

| | | |
|---|---|---|
| a) Growth factors | Transforming growth factor beta(TGFβ1), | 1-50 picoMolar. |
| preferably human. | fibroblast growth factor (FGF), vascular | |
| | endothelial cell growth factor (VEGF); | |
| | CTGF, | |
| | Insulin like growth factor I, Insulin like | |
| | Growth Factor II, Latency associated Peptide | |
| | of TGF-β1, effector of Mannose 6- | |
| | phosphate/IGF2 receptor. | |
| b) Extra cellular matrix | Fibronectin, Laminin, Collagens, Vitronectin | |
| proteins and their | or a suitable mixture of these. | |
| fragments containing | | |
| integrin | | |
| binding/activating | | |
| domains | | |
| c) Conditioned medium | Prepared from Mononuclear cells in presence | Used as such or |
| | of erythropoietin | with suitable steps |
| | 2 I.U. ml⁻¹, GM-CSF as described in example | of concentration or |
| | herein. | dilution determined |
| | | empirically. |

B. Chemical Hematomodulators

| | | |
|---|---|---|
| a) Agent that modulates | Diacyl glycerols or (—) Indolactam V, Farnesyl | 0.1 to 100 |
| the intracellular | thiotriazole, 12-O-tetra-decanoyl phorbol, 13- | microMolar. |
| Serine/Threonine protein | acetate, 1,6-bis(Cyclohexy-loximinocarbonyl | |
| kinases or protein kinase | amino) hexane, 8-4(-chloro-phenyl thio) | |
| C boosters | cGMP, 1,6- | |
| | bis(Cyclohexyloximinocarbonylamino) | |
| | hexane(U-57908), TGF-β1 mimetics, bFGF | |
| | receptor mimetics | |
| b) Boosters for cGMP- | 8-4-Chlorophenylthio) guanosine3′,5′-cyclic | 0.1 to 100 |
| activated signaling | monophosphate Sodium salt, Adenosine 3′, | microMolar |
| processes including the | 5′-cyclic monophosphothioate-Rp-isomer, | |
| protein kinases | Zaprinast and Sildenafil | |
| c) Focal adhesion kinase | Peptide such as Trp-Gln-Pro-Pro-Arg-Ala- | 0.1 to 100 |
| booster | Arg-Ile, linear or “head to tail cyclic peptides | microMolar |
| | such as “arg-Gly-Asp-Serine” | |
| d) Boosters of integrin | Peptides such as Trp-Gln-Pro-Pro-Arg-Ala- | 0.1 to 100 |
| linked kinase, PI3Kinase | Arg-Ile, linear or cyclic peptides comprising | microMolar |
| and Akt-Kinase | the sequence motif “Arg-Gly-Asp-Ser” and | |
| | the protein TGFβ1 | |
| e) Calcium signal | Thapsigargin, Cyclopiazonic acid and 8-(N, | 0.1 to 100 |
| modulators | N-diethylamino)octyl-3,4,5- | microMolar |
| | trimethoxybenzoate (TMB-8), Di Bromo | |
| | BAPTA and such other Calcium ion chelators | |
| f)Inhibitor of intracellular | 3-Amino-2,4,-dicyano-5-(3′,4,5′- | 1 to 100 |
| tyrosine kinase activity | trihydroxyphenyl) penta-2,4-dienonitrile | microMolar |
| | (Tyrphostin AG183/Tyrphostin A51). | |
| g) Inhibitor of bFGF | Ala-Pro-Ser-Gly-His-Tyr-Lys-Gly peptide | 0.1 to 100 |
| receptor function | | micromolar |
| h)Agents acting as | Nitric Oxide donors like S-Nitroso | 0.1 to 100 to |
| diffusible chemical | Penicillamine (SNP), 2-(N,N- | microMolar |
| signals. | Dimethylamino)-diazenolate-2-oxide | |
| | (DEANONOate), Stromal Cell Derived | |
| | Factor-1 alpha, Stromal Cell Derived Factor- | |
| | 1beta, effectors of CXCR4. | |
| Effectors of | Specific or non specific modulators of | 10 μMolar to |
| i)Integrin receptor, | integrins comprising α5:β1, α2:β1, α2b:β3, | 100 μMolar |
| j) FocalAdhesionKinase, | α4:β1, αv:β5, αv:β3, fibronectin adhesion | |
| k)bFGF-receptor. | promoting factor (FAK-activator), short | |
| | peptide integrin regulators containing linear, | |
| | cyclized or polymerized Arg-Gly-Asp-Ser, | |
| | bFGF regulator such as Ala-Pro-Ser-Gly- | |
| | His_Tyr-Lys-Gly, natural fibronectins or sub- | |
| | fragments of fibronectin containing various | |
| | integrin interacting domains, cell binding | |
| | domain, heparin binding domain and gelatin | |
| | binding domain. | |
| l) Dominant negative | Cyclo-1-Adamantane acetyl-Cys-Gly-Arg- | 10-100 micromolar. |
| inhibitor of integrin | Gly-Asp-Ser-Pro-Cys | |
| function | (cyclized between the two Cys at 1 and 8 | |
| | position). | |
| m) Agent or factor that | NO donors such as SNN, SNAP SNP, | 0.1 μM to 100 μM |
| promotes NO signaling | DEANONOate, and nitrates such as | 0.1-10 ng/ml. |
| and | isosorbide mononitrate, and the like. | |
| vasodilation. | | |
| n) Agent that promotes a | Transforming Growth Factor beta1, | 1-100 μM |
| hypoxic state in cells | N-oxalyl-D-alanine, N-oxalyl-L-alanine and | |
| under normoxic | N-oxalyl glycine. | |
| conditions | | |
| o) Agents that act | A poly (ADP-ribose) polymerase inhibitor, | 0.1 μM to 100 μM |
| through stem and | latency associated peptide of TGF beta 1, a | |
| progenitor cells to | soluble or cell surface associated mannose 6- | |
| promote their | phosphate containing glyco-conjugate, IGF-I | |
| proliferation and | and IGF-II, and effectors of their receptors, | |
| survival, termed as SPC | boosters of cGMP signaling. | |
| priming | | |
| hematomodulators. | | |

C. Immunological hematomodulator

| | | |
|---|---|---|
| An antibody reagent or | Activating type of antibodies to various alpha | 10 to 100 μg/ml |
| its functional | and beta subunits of integrins such as an | Or, sufficient to |
| homologues capable of | activating type of anti-beta 3 integrin | cause aggregation |
| activating adhesive | antibody. | of target cells to the |
| interactions on | | extent of 50% or |
| mesenchymal cells | | more. |
| through the integrin | | |
| receptors | | |

d. Combinatorial hematomodulator

| | | |
|---|---|---|
| Combination of above | Two or more hematomodulators selected from | As indicated above |
| types | the table above, used concomitantly as a | for specific types. |
| | mixture or used sequentially. | |

4. A composition as claimed in claim 1 wherein the mesenchymal cells are obtained from cord blood and placenta from a mammalian source, preferably of human origin.

5. A composition as claimed in claim 1 wherein the mesenchymal cells are obtained from iliac crest rib bones, femur bone or any other suitable bone specimen from a mammal, preferably of human origin.

6. A method for creation of an artificial bone marrow environment, comprising the steps of:
i) obtaining and growing mesenchymal cells in a growth medium suitable for mammalian cell culture preferably selected from Iscove's Modified Dulbecco's Medium (IMDM), Dulbecco's Modified Eagle Medium (DMEM), Alpha Minimum Essential Medium (MEM), RPMI-1640 supplemented with fetal bovine serum and optionally with methyl cellulose and erythropoietin,
ii) contacting the mesenchymal cells prepared in [i) above with a hematopoietic modulator or a plurality of modulators claimed in claim 3 for at least thirty minutes whereby hypoxic state is induced in the mesenchymal cells and are activated to form ABME,
iii) optionally, washing the ABME cells in step ii) to remove the hematomodulators,
iv) contacting cells of ABME created at step 7.iii) with SPC as such or optionally after treating them with priming hematomodulators, so as to activate hematopoiesis with features such as SPC-homing, SPC-engraftment, self renewal, and to robustly form blood cells as it occurs in a bone marrow like environment, in vitro,
v) optionally, processing the SPC obtained in step iv for one or more cycles of contact with fresh ABME to progressively expand SPC population and committed progenitors and to realize greater benefits.

7. (canceled)

8. A method to discover new biological, chemical or immunological entities for use as hematomodulators.

9. A method to compare plurality of tissue samples, capable of yielding mesenchymal cells, in their relative efficacy to form ABME in vitro.

10. A method to induce quiescence in SPC by using ABME prepared with suitable hematomodulators selected from claim 3.

11. A method to distinguish between normal and pathological SPC.

12. A method to purge leukemia progenitors from the bone marrow population of SPC.

13. A method to induce and sustain a hypoxic sate in mesenchymal cells under normoxic conditions.

14. A kit useful for creating an artificial bone marrow environment (ABME) for regulating blood cell formation in vitro comprising:
a) one or more hematopoietic modulator which may be a biological, a chemical or an immunological agent,
b) a diluent for hematomodulator comprising Dimethyl Sulfoxide, phosphate buffer, IMDM;
c) a medium suitable for culturing mesenchymal cells e.g. Dulbecco's medium, RPMI-1640, IMDM with growth supplements;
d) a wash solution useful in removing used hematomodulators such as phosphate buffered saline or IMDM;
e) a solution useful for harvesting the cells of ABME or/and recycling activated SPC for further use such as solutions comprising proteolytic enzymes, inhibitors and ethylenediamine tetraacetic acid(EDTA);
f) a solution of hematomodulators to prime the stem progenitor cells;
g) wash solutions to remove the priming agents before using the primed stem progenitor cells such as Phosphate buffered saline or IMDM,
h) a medium for in vitro blood cell formation comprising a supporting template or scaffold for ABME, cells of ABME, pro-hematopoietic growth, differentiation and survival factors, growth medium and optionally methylcellulose, serum and
i) manual of instructions.

15. A kit as claimed in claim 5 further comprising of a diluent for hematomodulator, a medium suitable for the contacting of hematomodulators with mesenchymal cells to create the ABME, solutions helpful to remove the used hematomodulators, solutions for harvesting cells of ABME and/or activated SPC, solutions of hematomodulators acting as priming agents to render SPC able to further synergise with ABME, wash solutions to remove the priming agents after their use, a medium for contacting primed SPC to the ABME for promoting in vitro engraftment, SPC activation, SPC self renewal and robust blood cell formation.

16-17. (canceled)
